(19) United States
(12) Patent Application Publication
(10) Pub. No.: US 2008/0058533 A1
(43) Pub. Date: Mar. 6, 2008

(54) PYRANOINDAZOLE CYCLIC CARBONATES AND METHODS OF USE

(75) Inventors: Pete DELGADO, Fort Worth, TX (US); Raymond E. CONROW, Crowley, TX (US)

Correspondence Address: ALCON, IP LEGAL, TB4-8, 6201 SOUTH FREEWAY, FORT WORTH, TX, 76134, US

(73) Assignee: ALCON, INC., Fort Worth, TX (US)

(21) Appl. No.: 11/847,561

(22) Filed: Aug. 30, 2007

Related U.S. Application Data
(60) Provisional application No. 60/824,151, filed on Aug. 31, 2006.

(51) Int. Cl. C07D 231/54

(52) U.S. Cl. 548/358.5

(57) ABSTRACT

Described are methods of making pyranoindazole compounds comprising converting a pyranoindazole diol mixture to form a diastereomeric mixture of cyclic pyranoindazole carbonates, separating the mixture of cyclic pyranoindazole carbonates, and converting at least one of the separated diastereoisomeric cyclic pyranoindazole carbonates by hydrogenolysis. Also disclosed are intermediates useful for such and additional methods.

### CROSS-REFERENCE TO RELATED APPLICATION

This application claims priority under 35 U.S.C. §119 to U.S. Provisional Patent Application No. 60/824,151 filed Aug. 31, 2006, the entire contents of which are incorporated herein by reference.

### TECHNICAL FIELD OF THE INVENTION

The present invention is directed to intermediates for the synthesis of pyranoindazole compounds. The invention is particularly directed to pyranoindazole cyclic carbonate intermediates and processes for producing such and additional intermediates.

### BACKGROUND OF THE INVENTION

5-HT₂ serotonergic receptor agonists are being investigated as compounds useful for treating a variety of disease states, including the ocular disease glaucoma. The disease state referred to as glaucoma is characterized by a permanent loss of visual function due to irreversible damage to the optic nerve. The several morphologically or functionally distinct types of glaucoma are typically characterized by elevated intraocular pressure (IOP), which is considered to be causally related to the pathological course of the disease. If glaucoma or ocular hypertension is detected early and treated promptly with medications that effectively reduce elevated intraocular pressure, loss of visual function or its progressive deterioration can generally be ameliorated. There is, therefore, a need for therapeutic agents that control IOP.

Pyranoindazole 5-HT₂ serotonergic receptor agonists have been disclosed as having utility as agents for treating glaucoma and elevated IOP in U.S. Pat. No. 6,696,476 to Chen et al., issued Feb. 24, 2004, the entire contents of which are herein incorporated by reference. It is an object of the present invention to provide additional intermediates and processes for the synthesis of pyranoindazoles. Other objects will be evident from the ensuing description and claims.

### SUMMARY OF THE INVENTION

The present invention is directed to processes and intermediates useful for the synthesis of pyranoindazole 5-HT₂ serotonergic receptor agonists. Some such pyranoindazole cyclic carbonate intermediates are useful for the synthesis of pyranoindazole 5-HT₂ serotonergic receptor agonists. Embodiments of the present invention provide efficient and simplified methods for the synthesis of such pyranoindazole compounds.

One embodiment of the present invention is a method of making a pyranoindazole comprising converting a pyranoindazole diol mixture to form a diastereomeric mixture of cyclic pyranoindazole carbonates, separating the mixture of cyclic pyranoindazole carbonates, and converting at least one of the separated diastereoisomeric cyclic pyranoindazole carbonates by hydrogenolysis.

Intermediates that are useful for the synthesis of pyranoindazole 5-HT₂ serotonergic receptor agonists comprise cyclic pyranoindazole carbonate compounds represented by the formulas

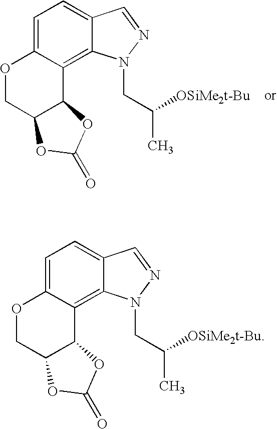

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. Figures provided herein are intended to help illustrate the invention or assist with developing an understanding of the invention, and are not intended to be definitions of the invention's scope.

### DETAILED DESCRIPTION OF THE INVENTION

The present invention relates to processes and intermediates for the synthesis of pyranoindazole 5-HT₂ serotonergic receptor agonists.

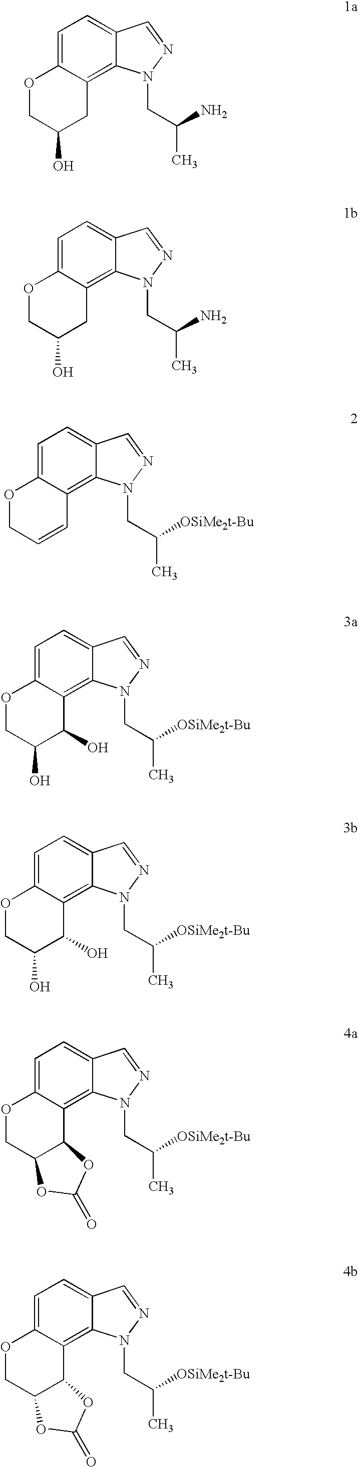

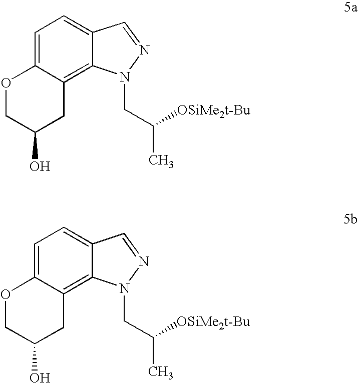

Compound 1a, (R)-1-((S)-2-aminopropyl) 1,7,8,9-tetrahydropyrano[2,3-g]indazol-8-ol, and compound 1b, (S)-1-((S)-2-aminopropyl) 1,7,8,9-tetrahydropyrano[2,3-g]indazol-8-ol, are members of a preferred class of pyranoindazole serotonergic agonists useful for the treatment of glaucoma as disclosed in U.S. Pat. No. 6,696,476.

Among the methods of synthesis of 1a (Example 4, Step C of U.S. Pat. No. 6,696,476) is the hydroboration of chromene 2 with 9-BBN which affords alcohols 5a and 5b plus benzylic alcohol regioisomers. The alcohol 5a is isolated from this mixture by tedious column chromatography. Further processing as disclosed in U.S. Pat. No. 6,696,476 serves to convert 5a to 1a. This sequence is not suitable for scaleup synthesis because of the difficulty of the chromatographic separation necessary to deliver either pure 5a or pure 5b.

U.S. Pat. No. 6,696,476 also discloses (Example 7, Step D) the dihydroxylation of chromene 2 to give diols 3a and 3b. In that case, asymmetric dihydroxylation, wherein a chiral catalyst is used to favor the formation of one of two stereoisomeric diols, was used. Certain asymmetric dihydroxylation processes are known: U.S. Pat. Nos. 5,516,929 and 5,260,461; McKee et al., Organic Syntheses Vol. 70:47, 1992; Sharpless et al., Journal of Organic Chemistry, Vol. 57:2768, 1992; Ahrgren et al., Organic Process Research and Development, Vol. 1:425, 1997; Xie, et al., Journal of Medicinal Chemistry 42:2662, 1999. Additionally, the dihydroxylation of alkenes without the use of a chiral catalyst is known: VanRheenen et al., Organic Syntheses Collective Vol. 6:342, 1988.

In certain embodiments of the present invention, use is made of the unexpected finding by the inventors that the cyclic carbonates 4a and 4b exhibit sufficiently different chromatographic polarity to enable their practical separation on a multihundred gram scale. Hydrogenolysis via known processes (see, e.g., Sprott et al., Organic Letters, Vol. 5:2465, 2003) of the separated carbonates 4a and 4b then affords compounds 5a and 5b.

One embodiment of the present invention is a method of making a pyranoindazole comprising converting a pyranoindazole diol mixture to form a diastereomeric mixture of cyclic pyranoindazole carbonates, separating the mixture of cyclic pyranoindazole carbonates, and converting at least one of the separated diastereoisomeric cyclic pyranoindazole carbonates by hydrogenolysis. In a preferred embodiment of the present invention, the pyranoindazole diol mixture comprises 3a and 3b, which is converted to form a mixture of 4a and 4b. 4a and 4b may be separated using techniques described herein or known to those of skill in the art, and at least one of the separated moieties are then converted by hydrogenolysis to form 5a and/or 5b. 5a and/or 5b may be further reacted in certain embodiments to form a serotonergic agonist using methods described herein, described in U.S. Pat. No. 6,696,476, or known to those of skill in the art.

Specific reaction conditions for the above-described processes can be readily ascertained by those of skill in the art using the information presented above together with conditions provided below in the Examples.

### EXAMPLES

### Example 1

(a) N-Methylmorpholine N-oxide (170 mL of a 50% aqueous solution) was diluted with 85 mL of water and 110 mL of tert-butyl alcohol. Hydroquinine 2,5-diphenyl-4,6-pyrimidinediyl diether ((DHQ)₂PYR, 4.14 g) was added and the mixture was stirred until all the solid dissolved and was then placed under a nitrogen atmosphere. Potassium osmate dihydrate (1.58 g) was then added and the mixture was stirred until all of the solid dissolved. To this solution at ambient temperature was added, over a four hour period, a solution of chromene 2 (220 g) in 650 mL of tert-butyl alcohol. After stirring overnight, ethyl acetate (1.3 L) was added, followed by a solution of 80 g of sodium sulfite in 1.3 L of water. The mixture was stirred vigorously for 0.5 h. The aqueous phase was separated and extracted twice with 2-L portions of ethyl acetate. The combined organic solution was washed with 0.5 L of water and with 0.5 L of saturated aqueous KH₂PO₄, dried over sodium sulfate, eluted through a pad of Florisil with ethyl acetate, and concentrated in vacuo to give 239 g of an oil which contained a 15:1 mixture of diols 3a and 3b.

(b) To a stirred solution of the foregoing mixture of diols 3a and 3b in 2.3 L of dichloromethane was added 125 g of 1,1′-carbonyldiimidazole followed by 36.3 g of 4-dimethylaminopyridine. After 4.5 h, a solution of 82 g of KH₂PO₄ in 1 L of water was added and the mixture was stirred vigorously for 0.5 h. The aqueous phase was separated and extracted with dichloromethane. The combined organic solution was concentrated to give a mixture of carbonates 4a and 4b. This mixture was purified by chromatography on silica, eluting with a gradient of 9% to 14% ethyl acetate in heptane, to give 210 g of carbonate 4a.

(c) A solution of 1012 g of carbonate 4a in 16 L of ethanol was purged with nitrogen. Ammonium formate (790 g) was added followed by 101 g of 5% palladium on calcium carbonate. Ethanol (4 L) was added, followed by 5 g of 10% palladium on carbon (wet weight, ½ water). The mixture was stirred until the starting carbonate was consumed by TLC, then filtered through Celite eluting with ethanol. The filtrate was concentrated in vacuo. Water (16 L) was added and the mixture was extracted three times with 10-L portions of ethyl acetate. The combined organic extract was dried over sodium sulfate, filtered, concentrated and the residue was purified by chromatography on silica eluting with a gradient of 25% to 50% ethyl acetate in heptane. The purified product was recrystallized from 8 L of heptane to give 614 g of alcohol 5a as a solid.

### Example 2

(a) The dihydroxylation procedure of Example 1 (a) was followed, using 4.0 mL of 50% aqueous N-methylmorpholine N-oxide, 15 mL of tert-butyl alcohol, 4 mL of water, 37 mg of potassium osmate dihydrate, 87 mg of hydroquinine 1,4-phthalazinediyl diether ((DHQ)₂PHAL) and 5.0 g of chromene 2, yielding 5.57 g of a 5:1 mixture of diols 3a and 3b.

(b) The foregoing 5:1 mixture of diols 3a and 3b (5.5 g) was reacted according to the procedure of Example 1(b), using 3.0 g of 1,1′-carbonyldiimidazole, 0.90 g of 4-dimethylaminopyridine and 45 mL of dichloromethane to give, after chromatography on silica eluting with 1:3 ethyl acetate/hexane, 4.56 g of carbonate 4a followed by 0.87 g of carbonate 4b.

(c) Carbonate 4a may be used in a hydrogenolysis process similar to that described in Example 1 (a) above.

(d) Palladium, 5% on CaCO₃, 0.72 g, was added to a rapidly stirred solution of carbonate 4b (6.0 g, 14.8 mmol) and ammonium formate (10.0 g, 159 mmol) in 120 mL of absolute EtOH under a nitrogen atmosphere. After stirring for 16 h at RT, the mixture was filtered and the solids were rinsed well with EtOAc and with water. The filtrate was further partitioned between EtOAc and water. The organic solution was dried (MgSO₄), filtered and concentrated in vacuo to give 5.73 g of 5b as an oil.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

We claim:

1. A compound of the formula:

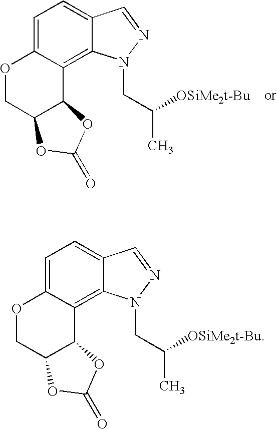

2. A method of making a pyranoindazole comprising:
a) converting a pyranoindazole diol mixture to form a diastereomeric mixture of cyclic pyranoindazole carbonates;
b) separating said mixture of cyclic pyranoindazole carbonates; and
c) converting at least one of the separated diastereoisomeric cyclic pyranoindazole carbonates by hydrogenolysis.

3. The method of claim 2 wherein said pyranoindazole diol mixture comprises

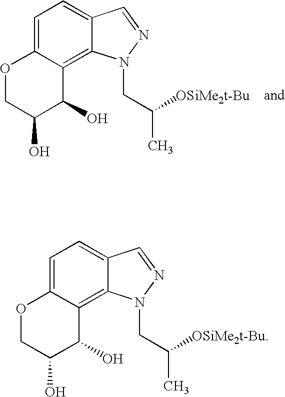

4. The method of claim 2 wherein said formed mixture comprises

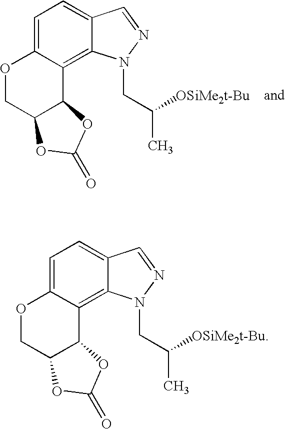

5. The method of claim 2 wherein said hydrogenolysis forms

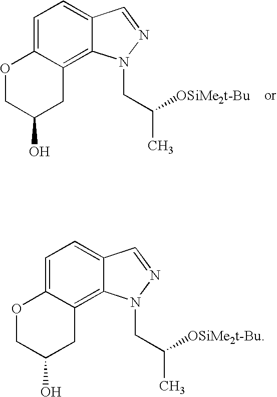

6. The method of claim 2 further comprising:
reacting the pyranoindazole formed by said converting by hydrogenolysis to form a serotonergic agonist.

7. The method of claim 6 wherein said reacting forms a serotonergic agonist of formula 1a or 1b: [chemical structure]
